(19) United States
(12) Patent Application Publication
(10) Pub. No.: US 2020/0121167 A1
(43) Pub. Date: Apr. 23, 2020

(54) INSERTION DEVICE

(71) Applicant: OLYMPUS CORPORATION, Tokyo (JP)

(72) Inventor: Kohei ARAKI, Tokyo (JP)

(73) Assignee: OLYMPUS CORPORATION, Tokyo (JP)

(21) Appl. No.: 16/697,862

(22) Filed: Nov. 27, 2019

Related U.S. Application Data
(63) Continuation of application No. PCT/JP2018/007269, filed on Feb. 27, 2018.

(30) Foreign Application Priority Data
May 31, 2017 (JP) 2017-107539

(51) Int. Cl. A61B 1/005; A61B 1/00; A61B 1/05

(52) U.S. Cl. CPC A61B 1/0055; A61B 1/05; A61B 1/00098

(57) ABSTRACT

An endoscope as an insertion device includes an insertion portion configured to be inserted into a subject from a distal end side in a longitudinal axis direction. The insertion portion includes: a distal end portion; a bending portion provided on a proximal end side of the distal end portion and configured to bend in a first direction according to a bending operation by an operator; a passive bending portion as a first flexible tube portion provided on a proximal end side of the bending portion and configured to passively bend by receiving an external force without being bent by the bending operation by the operator; and a corrugated tube as a second flexible tube portion provided on a proximal end side of the passive bending portion. The passive bending portion has higher bending rigidity in a second direction orthogonal to the first direction than in the first direction.

### CROSS REFERENCE TO RELATED APPLICATION

This application is a continuation application of PCT/JP2018/007269 filed on Feb. 27, 2018 and claims benefit of Japanese Application No. 2017-107539 filed in Japan on May 31, 2017, the entire contents of which are incorporated herein by this reference.

### BACKGROUND OF INVENTION

### 1. Field of the Invention

The present invention relates to an insertion device, and more particularly, to an insertion device including an insertion portion provided with a bending portion on a distal end side.

### 2. Description of the Related Art

Conventionally, insertion devices such as endoscopes have been widely used. The endoscopes can perform in-vivo examination of a subject in such a manner that an insertion portion of the endoscope is inserted into the subject to obtain in-vivo images of the subject and endoscopic images are displayed on a display apparatus. A bending portion, that is, an active bending portion is provided on a distal end side of the insertion portion of the insertion device so as to be freely bendable by a user's operation.

Further, International Publication No. WO 2011/136115 also discloses an endoscope including a passive bending portion that is provided on a proximal end side relative to the active bending portion to be freely bent by an operation and passively bends due to an applied external force. The passive bending portion is bendable in up, down, left, and right directions with respect to an insertion direction of an insertion portion according to the applied external force. By the passive bending portion provided in this way, a radius of curvature of the bending portion is increased, and the insertion portion satisfactorily passes into a flexure portion in the subject.

In general, for example, during insertion into a large intestine having mobility, an operator usually bends the active bending portion of the endoscope in an up-down direction while seeing the endoscopic image displayed on the display apparatus and inserts the insertion portion. When the insertion portion is pushed in and passes through the flexure portion of the large intestine which is the subject, the operator allows the insertion portion to pass through the flexure portion by bending the active bending portion upward, for example.

For example, when the operator of the endoscope inserts the insertion portion into the sigmoid colon, the transverse colon or the like, the operator hooks the active bending portion to the flexure portion of the sigmoid colon or the like by bending the active bending portion upward. The operator pulls the insertion portion forward while hooking the active bending portion, subsequently swings the insertion portion in a predetermined direction around an axis of the insertion portion to convolute the flexure portion of the large intestine and straighten the flexure portion, and then pushes the insertion portion deeply.

When the insertion portion is swung, a swinging force is applied to the active bending portion, and thereby the active bending portion rises up and the large intestine is convoluted without being stretched. As a result, the insertion portion is easily inserted, and the burden and pain on the patient are reduced. The operator inserts the insertion portion into the large intestine deeply while aligning the center axis of the large intestine with the center axis of the insertion portion.

### SUMMARY OF THE INVENTION

An insertion device according to an aspect of the present invention is an insertion device including an insertion portion configured to be inserted into a subject from a distal end side in a longitudinal axis direction, the insertion portion including: a distal end portion provided at a distal end of the insertion portion; a bending portion provided on a proximal end side of the distal end portion and configured to bend in a first direction according to a bending operation by an operator; a passive bending portion provided on a proximal end side of the bending portion and configured to passively bend by receiving an external force without being bent by the bending operation by the operator; and a flexible tube portion provided on a proximal end side of the passive bending portion and having flexibility, wherein the passive bending portion includes a plurality of annular bending pieces connected in series, and two adjacent bending pieces in the plurality of bending pieces are coupled to each other on either of a first turning axis inclined by a first angle being in a range of from an angle greater than 0° to an angle less than +45° in a second direction orthogonal to the first direction around a center axis of the insertion portion or a second turning axis inclined by a second angle being in a range of from an angle greater than −45° to an angle less than 0° when the insertion portion is viewed from the distal end side, so that the passive bending portion has higher bending rigidity in the second direction than in the first direction.

An insertion device according to another aspect of the present invention is an insertion device including an insertion portion configured to be inserted into a subject from a distal end side in a longitudinal axis direction, the insertion portion including: a distal end portion provided at a distal end of the insertion portion; a bending portion provided on a proximal end side of the distal end portion and configured to bend in a first direction and a second direction orthogonal to the first direction when the insertion portion is viewed from the distal end side, according to a bending operation by an operator; a passive bending portion provided on a proximal end side of the bending portion and configured to passively bend by receiving an external force without being bent by the bending operation by the operator; a flexible tube portion provided on a proximal end side of the passive bending portion and having flexibility; and an image acquisition apparatus provided at the distal end portion and configured to pick up an image of the subject, wherein the passive bending portion includes a plurality of annular bending pieces connected in series, two adjacent bending pieces in the plurality of bending pieces are coupled to each other on either of a first turning axis inclined by a first angle being in a range of from an angle greater than 0° to an angle +30° in the second direction around a center axis of the insertion portion or a second turning axis inclined by a second angle being in a range of from −30° to an angle less than 0° when the insertion portion is viewed from the distal end side, so that the passive bending portion has higher bending rigidity in the second direction than in the first direction, an inclined angle of the first turning axis and an inclined angle of the second turning axis are set to be equal to each other in the second direction, and the second direction is parallel to a left-right direction orthogonal to an up-down direction of an image obtained by the image acquisition apparatus and displayed on a screen of a display apparatus.

### BRIEF DESCRIPTION OF THE DRAWINGS

FIG. 1 is a schematic diagram of an endoscope 1 according to a first embodiment of the present invention;

FIG. 2 is a partial cross-sectional view of a distal end portion provided in an insertion portion 2 of the endoscope 1 according to the first embodiment of the present invention;

FIG. 3 is a cross-sectional view of an active bending portion 14 provided in the insertion portion 2 of the endoscope 1 according to the first embodiment of the present invention;

FIG. 4 is a partial cross-sectional view of a passive bending portion 15 provided in the insertion portion 2 of the endoscope 1 according to the first embodiment of the present invention;

FIG. 5 is a cross-sectional view of the passive bending portion 15 taken along line V-V in FIG. 4;

FIG. 6 is a perspective view of a plurality of bending pieces of the passive bending portion 15 which is in a straight state according to the first embodiment of the present invention;

FIG. 7 is a perspective view of the plurality of bending pieces of the passive bending portion 15 which is in a bending state according to the first embodiment of the present invention;

FIG. 8 is a view for describing a bendable range of the passive bending portion 15 according to the first embodiment of the present invention;

FIG. 9 is a view schematically showing distribution of the maximum bending angle of the passive bending portion 15 according to the first embodiment of the present invention;

FIG. 10 is a view showing a component force applied to a turning axis when pressing of the large intestine is applied to the passive bending portion 15 from an up-direction side according to the first embodiment of the present invention;

FIG. 11 is a view showing a component force applied to the turning axis when pressing of the large intestine is applied to the passive bending portion 15 from a right-direction side according to the first embodiment of the present invention;

FIG. 12 is a view for describing an operation of inserting the insertion portion 2 into the large intestine using the endoscope of the present embodiment, according to the first embodiment of the present invention;

FIG. 13 is a view for describing an operation of inserting the insertion portion 2 into the large intestine using the endoscope of the present embodiment, according to the first embodiment of the present invention;

FIG. 14 is a view for describing an operation of straightening the large intestine using the endoscope of the present embodiment, according to the first embodiment of the present invention;

FIG. 15 is a view for describing the operation of straightening the large intestine using the endoscope of the present embodiment, according to the first embodiment of the present invention;

FIG. 16 is a view for describing a case where mobility of the large intestine is lowered, according to the first embodiment of the present invention;

FIG. 17 is a view for describing a case where mobility of the large intestine is lowered, according to the first embodiment of the present invention;

FIG. 18 is a cross-sectional view of a passive bending portion 15A as viewed from a distal end side of an insertion portion 2 according to a second embodiment of the present invention;

FIG. 19 is a perspective view of a plurality of bending pieces of the passive bending portion 15A which is in a straight state according to the second embodiment of the present invention;

FIG. 20 is a schematic diagram of an endoscope 1A according to a third embodiment of the present invention;

FIG. 21 is a cross-sectional view of a flexible tube portion 12A taken along a center axis O in an up-down direction according to the third embodiment of the present invention;

FIG. 22 is a cross-sectional view of the flexible tube portion 12A taken along line XXII-XXII in FIG. 21;

FIG. 23 is a cross-sectional view of the flexible tube portion 12A taken along line XXIII-XXIII in FIG. 21;

FIG. 24 is a cross-sectional view of the flexible tube portion 12A taken along line XXIV-XXIV in FIG. 21;

FIG. 25 is a cross-sectional view of the flexible tube portion 12A taken along line XXII-XXII in FIG. 21 according to Modification 1 of the third embodiment of the present invention;

FIG. 26 is a flowchart showing an example of a procedure in which straightening of a flexure portion of a subject is performed;

FIG. 27 is a view showing an example of a state of the insertion portion inserted into the large intestine;

FIG. 28 is a view showing an example of a state of the insertion portion inserted into the large intestine;

FIG. 29 is a view showing an example of a state of the insertion portion inserted into the large intestine;

FIG. 30 is a view showing an example of a state of the insertion portion inserted into the large intestine;

FIG. 31 is a view showing an example of a state of the insertion portion inserted into the large intestine; and

FIG. 32 is a view showing an example of a state of the insertion portion inserted into the large intestine;

### DETAILED DESCRIPTION OF THE PREFERRED EMBODIMENTS

Hereinafter, an embodiment of the present invention will be described with reference to the drawings. Note that the drawings are schematic, attention should be paid to that the relationships of the thicknesses and the widths of the respective members, the ratios of the thicknesses of the respective members and the like differ from the actual relationships, ratios and the like, and among the drawings, the parts in which the mutual relationships and ratios of the dimensions differ are included as a matter of course.

(Configuration of Entire Endoscope)

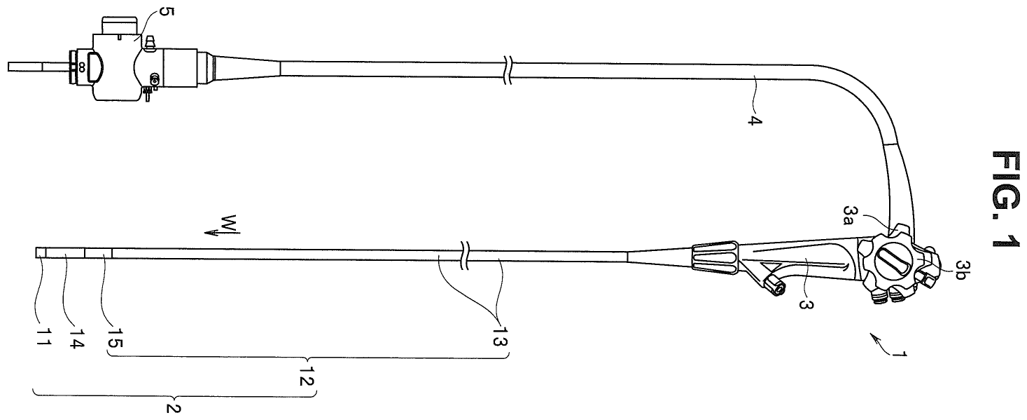

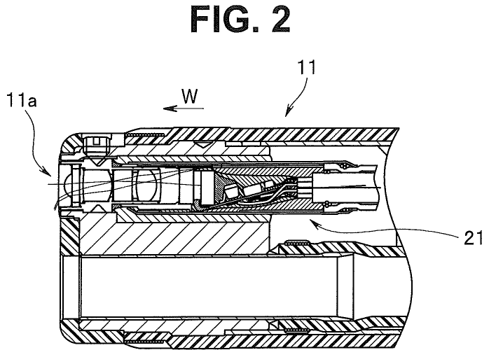

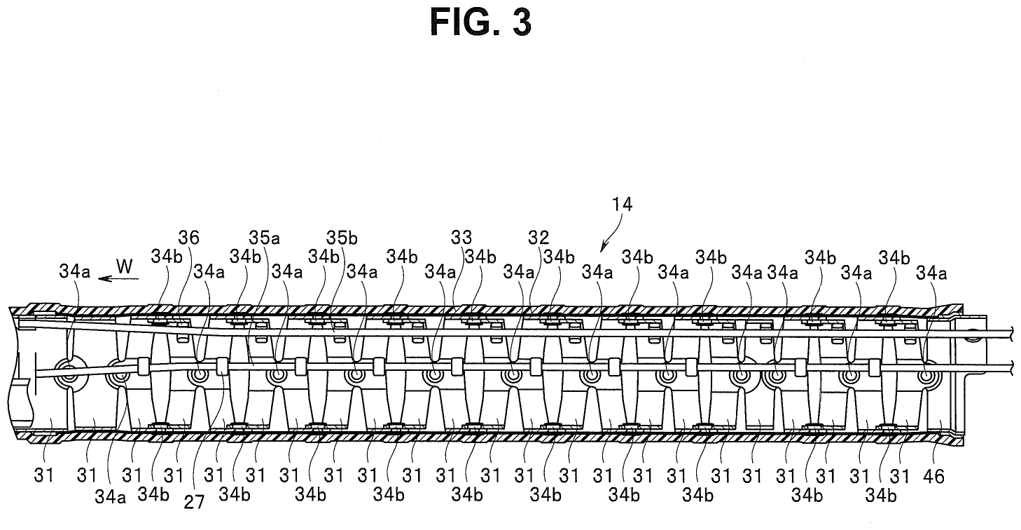

FIG. 1 is a schematic view of an endoscope 1 according to the present embodiment. FIG. 2 is a partial cross-sectional view of a distal end portion provided in an insertion portion 2 of the endoscope 1 of FIG. 1. FIG. 3 is a cross-sectional view of an active bending portion 14 provided in the insertion portion 2 of the endoscope 1 of FIG. 1.

As shown in FIG. 1, the endoscope 1 is configured by main parts including an insertion portion 2 to be inserted into a subject, an operation portion 3 connected consecutively to a proximal end side of the insertion portion 2, a universal cord 4 extending from the operation portion 3, and a connector provided in an extension end of the universal cord 4. The endoscope 1 is electrically connected to an external apparatus such as a control apparatus or an illumination apparatus, via the connector 5.

The operation portion 3 includes an upward/downward bending operation knob (hereinafter, simply referred to as a knob) 3a and a leftward/rightward bending operation knob (hereinafter, simply referred to as a knob) 3b that bend an active bending portion 14 which will be described below.

As shown in FIG. 2, inside a distal end portion 11, an image pickup unit 21 configured to observe the interior of a subject, an illumination unit (not shown) configured to illuminate the interior of the subject, and the like are provided. The image pickup unit 21 is provided on a rear side of an observation window 11a of the distal end portion 11.

In other words, the image pickup unit 21 is provided on the distal end side in a longitudinal axis direction from the active bending portion 14, as an image acquisition apparatus configured to pick up an image of the subject.

The insertion portion 2 includes a distal end portion 11, an active bending portion 14, and a flexible tube portion 12 in order from a distal end, and is formed in a thin and long shape in an insertion direction W. The insertion portion 2 is configured to be insertable into the subject from the distal end side in a longitudinal axis direction of the insertion portion 2.

The flexible tube portion 12 includes a passive bending portion 15 that is a first flexible tube portion and a corrugated tube 13 that is a second flexible tube portion, in order from the distal end.

(Configuration of Corrugated Tube)

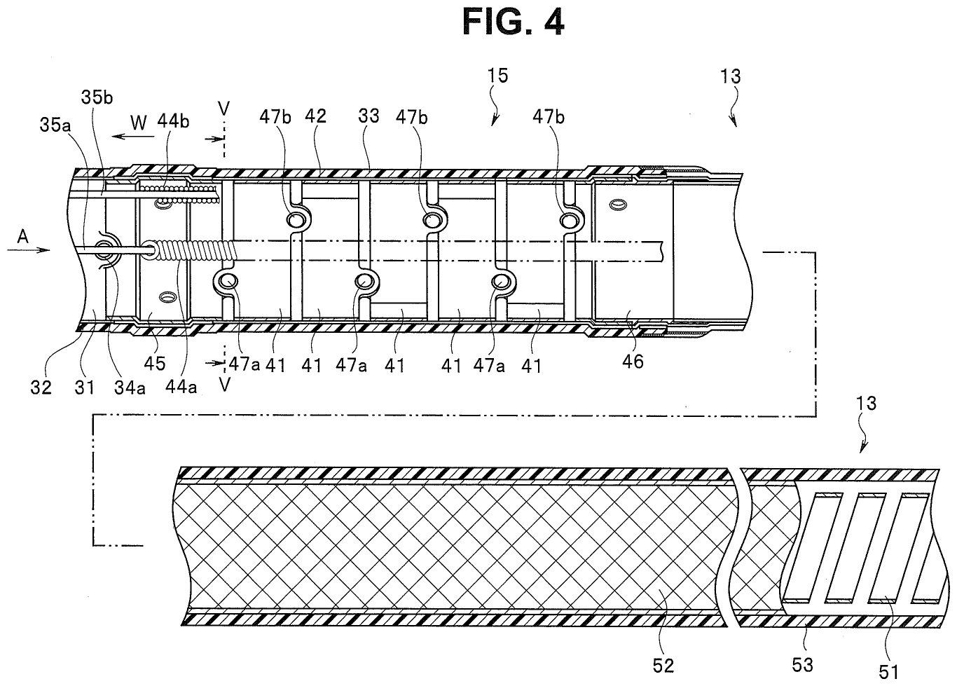

As shown in FIG. 4 to be described below, the corrugated tube 13 has a hollow shape, and, as will be described below, includes a helical tube 51 formed by helically winding a strip-shaped element member such as a thin plate member, a mesh-like mesh tube 52 that is provided on an outer peripheral side (on an outer peripheral surface) of the helical tube 51 and formed in a tubular shape by weaving fibers such as metal and resin, and an outer cover 53 that is provided on an outer peripheral side (on an outer peripheral surface) of the mesh tube 52 and has flexibility.

### (Configuration of Active Bending Portion)

The active bending portion 14 is bendable by 360° in an up-down direction as a first direction, a left-right direction as a second direction, and further in a direction in four directions of up, down, left, and right directions are combined depending on an operator's bending operation (here, operation of the knobs 3a and 3b) such as pulling or slackening of bending wires 35a to 35d (in FIG. 3, the bending wires 35c and 35d are not shown), which will be described below, to be inserted into the insertion portion 2. In other words, the active bending portion 14 can be bent in the left-right direction depending on an operator's bending operation when the insertion portion 2 is viewed from the distal end side.

More specifically, as shown FIG. 3, the active bending portion 14 is configured by a main part including a plurality of bending pieces 31, a braid 32 that covers outer peripheries of the plurality of bending pieces 31, and an outer coating resin 33 that coats an outer periphery of the braid 32. Each of the bending pieces 31 has an annular shape and is made of metal such as stainless steel.

Here, the up-down direction is an up-down direction of a screen of the display apparatus on which an endoscopic image obtained by an image pickup of the image pickup unit 21 is displayed, and the left and right direction is a left and right direction of the screen of the display apparatus on which the obtained endoscopic image is displayed.

As shown in FIG. 3, the plurality of bending pieces 31 are coupled to each other so that each of the bending pieces 31 can turn around a predetermined turning axis in an insertion direction W (a distal end direction of the insertion portion 2). In other words, two bending pieces 31 adjacent to each other in insertion direction W are turnably coupled to each other by a plurality of rivets 34a and 34b configuring turning axes located at positions different from each other by 90° in a circumferential direction J of the bending pieces 31.

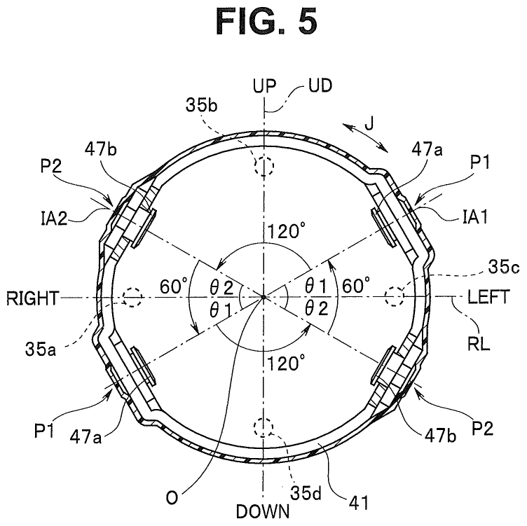

More specifically, the bending pieces 31 adjacent to each other in the insertion direction W are turnably coupled to each other in the up-down direction by two opposing rivets 34a (only one rivet being shown in FIG. 3), and are turnably coupled to each other in the left and right direction by two rivets 34b opposite to each other at positions different from the rivets 34a by 90° in the circumferential direction J. The two rivets 34a configure a first turning axis RL (defined in FIG. 5), and the two rivets 34b configure a second turning axis UD (defined in FIG. 5).

As shown in FIG. 3, the bending pieces 31 adjacent to each other are alternately coupled by the rivets 34a and 34b in such a manner that, for example, when the first bending piece 31 and the second bending piece 31 among the bending pieces 31 are coupled to each other by the rivet 34a, the second bending piece 31 and the third bending piece 31 are coupled to each other by the rivet 34b, the third bending piece 31 and the fourth bending piece 31 are further coupled to each other by the rivet 34a and so on.

Accordingly, the active bending portion 14 is configured to be bendable by 360° in the four directions of up, down, left, and right directions and in the direction in which the four directions of up, down, left, and right directions are combined. In other words, the active bending portion 14 is bendable in a plurality of directions by the first turning axis RL and the second turning axis UD shown in FIG. 5.

As shown in FIG. 3, the four bending wires 35a to 35d (only the bending wires 35a and 35b being shown in FIG. 3), which are located differently from each other by 90° in the circumferential direction J of the bending pieces 31, are inserted into the active bending portion 14. The two bending wires 35a and 35c are located in the same position with the two rivets 34a in the circumferential direction J along a center axis of the insertion portion 2. The two bending wires 35b and 35d are located in the same position with the two rivets 34b in the circumferential direction J along the center axis of the insertion portion 2.

Furthermore, the four bending wires 35a to 35d are supported by wire receivers 36 provided on the respective bending pieces 31 in the active bending portion 14, and the distal ends of the respective wires 35a to 35d are connected to the bending piece 31, which is located at the outermost distal end side in the insertion direction W, among the plurality of bending pieces 31. As a result, as the bending wires 35a to 35d are pulled and slackened, the respective bending pieces 31 turn around the turning axis of either the rivet 34a or the rivet 34b, and the active bending portion 14 is bent.

As described above, the active bending portion 14 configures the bending portion that is bendable in the up, down, left, and right directions according to the bending operation by the operator.

(Configuration of Passive Bending Portion)

The passive bending portion 15, which is the first flexible tube portion, is provided between the active bending portion 14 and the corrugated tube 13 which is the second flexible tube portion. In other words, the passive bending portion 15 is the first flexible tube portion provided on the distal end side of the corrugated tube 13, which is the second flexible tube portion, at the proximal end side relative to the active bending portion 14.

The passive bending portion 15 cannot be bent according to a bending operation by the operator, but is passively bendable by 360° when an external force is applied to the passive bending portion 15 in four directions of up, down, left, and right directions, or in directions in which the four directions of up, down, left, and right directions are combined. In other words, the passive bending portion 15 is configured to be passively bendable without being actively bent by bending wires or other bending operation means.

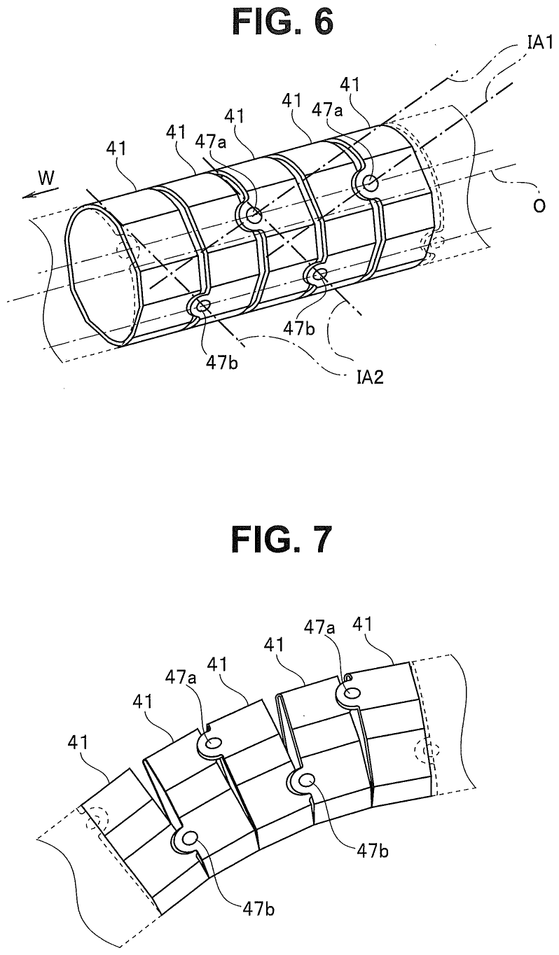

FIG. 4 is a partial cross-sectional view of the passive bending portion 15 provided in the insertion portion of the endoscope in FIG. 1. FIG. 5 is a cross-sectional view of the passive bending portion 15 taken along line V-V in FIG. 4. FIG. 5 is a view as viewed in a direction of an arrow A in FIG. 4. FIG. 6 is a perspective view of a plurality of bending pieces of the passive bending portion 15 which is in a straight state. FIG. 7 is a perspective view of the plurality of bending pieces of the passive bending portion 15 which is in a bending state.

As shown in FIG. 4, the passive bending portion 15 is configured by main parts including a plurality of bending pieces 41, a braid 42 that covers outer peripheries of the plurality of bending pieces 41, and an outer coating resin 33 that coats an outer periphery of the braid 42. Each of the bending pieces 41 has an annular shape and is made of metal such as stainless steel. In other words, the passive bending portion 15 includes the plurality of annular bending pieces 41 that are coupled in series.

The four bending wires 35a to 35d described above are inserted into the plurality of bending pieces 41 of the passive bending portion 15. The outer peripheries of the four bending wires 35a to 35d are covered with known coil pipes 44a to 44d (coil pipes 44c and 44d being not shown in FIG. 4). Distal ends of the coil pipes 44a to 44d are fixed to a pipe sleeve 45, which will be described below, by welding or the like.

The passive bending portion 15 includes the plurality of bending pieces 41. The plurality of bending pieces 41 are coupled in the insertion direction W so that the passive bending portion 15 is bendable. The plurality of bending pieces 41 are configured such that two adjacent bending pieces 41 in the insertion direction W are coupled to each other by two rivets provided at predetermined positions in the circumferential direction J of the respective bending pieces 41.

Specifically, as shown in FIG. 5, an axis passing in the left-right direction through the center axis O of the insertion portion 2 when the passive bending portion 15 is viewed from the distal end side of the insertion portion 2 indicates the first turning axis RL of the active bending portion 14 by which the active bending portion 14 is bent in the up-down direction due to the pulling and slackening of the two bending wires 35b and 35d.

Similarly, an axis passing in the up-down direction through the center axis O of the insertion portion 2 when the passive bending portion 15 is viewed from the distal end side of the insertion portion 2 indicates the second turning axis UD of the active bending portion 14 by which the active bending portion 14 is bent in the left-right direction due to the pulling and slackening of the two bending wires 35a and 35c.

The bending piece 31 on the most proximal end of the active bending portion 14 is connected to the bending piece 41 on the most distal end of the passive bending portion 15 via the pipe sleeve 45. The bending piece 41 on the most proximal end of the passive bending portion 15 is connected to the distal end portion of the corrugated tube 13 via the pipe sleeve 46.

As shown in FIG. 4, the active bending portion 14 and the passive bending portion 15 are connected to each other via the pipe sleeve 46 in a state where the braids 32 and 42 are covered on the outer peripheries of the respective bending pieces 31 and 41 before being coated with the outer coating resin 33.

In the passive bending portion 15, the two adjacent bending pieces 41 in the insertion direction W are coupled to each other at two positions P1 on a third turning axis IA1, which is inclined by a predetermined first angle θ1 (here, 30°) with respect to the first turning axis RL in a counterclockwise direction around the center axis O of the insertion portion 2, by two rivets 47a, or at two positions P2 on a fourth turning axis IA2, which is inclined by a predetermined second angle θ2 (here, 30°) with respect to the first turning axis RL in a counterclockwise direction around the center axis O of the insertion portion 2, by two rivets 47b.

In other words, the third turning axis IA1 is inclined by the predetermined first angle θ1 (here, 30°) with respect to the first turning axis RL when the passive bending portion 15 is viewed from the distal end side of the insertion portion 2. The fourth turning axis IA2 is inclined by the predetermined second angle θ2 (here, −30°) with respect to the first turning axis RL. As shown in FIG. 6, two third turning axes IA1 exist and two fourth turning axes IA2 also exist.

Then, the two bending pieces 41 coupled by the two rivets 47a are coupled to the bending pieces 41 adjacent to each other on the distal end side and the proximal end side at positions on the fourth turning axis IA2, which is turned by 120° in the counterclockwise direction around the center axis O when the passive bending portion 15 is viewed from the distal end side of the insertion portion 2, by two rivets 47b.

In other words, the two bending pieces 41 coupled by the two rivets 47b are coupled to the bending pieces 41 adjacent to each other on the distal end side and the proximal end side at positions on the third turning axis IA1, which is turned by 60° in the counterclockwise direction around the center axis O when the passive bending portion 15 is viewed from the distal end side of the insertion portion 2, by two rivets 47a.

The plurality of bending pieces 41 of the passive bending portion 15 are coupled with such a coupling relationship.

The two bending pieces 41 coupled by the rivet 47a are movable around the third turning axis IA1, and the two bending pieces 41 by the rivet 47b are movable around the fourth turning axis IA2.

As shown in FIG. 4, the plurality of bending pieces 41 are coupled to one another so that the two positions P1 and the two positions P2 in the passive bending portion 15 are alternately disposed from the distal end of the passive bending portion 15. As shown in FIG. 4 and the like, the bending piece 41 on the most distal end is coupled to the second bending piece 41 by the rivet 47b, the second bending piece 41 is coupled to the third bending piece 41 by the rivet 47a, the third bending piece 41 is coupled to the fourth bending piece 41 by the rivet 47b, and the fourth bending piece 41 is coupled to the fifth bending piece 41 by the rivet 47a.

The coupling of two adjacent bending pieces 41 on the third turning axis IA1 and the coupling of two adjacent bending pieces 41 on the fourth turning axis IA2 are alternately performed in the longitudinal axis direction.

The bending piece 41 on the most distal end of the passive bending portion 15 is coupled to the pipe sleeve 45 by two rivets 47a at two positions P1. The bending piece 41 on the most proximal end of the passive bending portion 15 is coupled to the pipe sleeve 46 by two rivets 47b at two positions P2.

Here, the angles θ1 and 02 are defined as 30° and −30°, respectively. However, when the counterclockwise angle around the center axis O is positive, the angle θ1 may be in a range of from an angle greater than 0° to an angle less than +45°, and the angle θ2 may be in a range of from an angle greater than −45° to an angle less than 0°. However, preferably, the angle θ1 is in a range of from an angle greater than 0° to an angle less than +30°, and the angle θ2 is in a range of from an angle greater than −30° to an angle less than 0°.

In other words, the two adjacent bending pieces 41 in the plurality of bending pieces 41 of the passive bending portion 15 are coupled to each other at positions on either of the third turning axis IA1 inclined by the angle in the range of from an angle greater than 0° to an angle less than +45° or the fourth turning axis IA2 inclined by the angle in the range of from an angle greater than −45° to an angle less than 0° with respect to the first turning axis RL around the center axis O of the insertion portion 2 when the insertion portion 2 is viewed from the distal end side.

In other words, as shown in FIG. 5, when the passive bending portion 15 is viewed from the distal end side of the insertion portion 2, the third turning axis IA1 is inclined by θ3 (=90°−θ1) with respect to the second turning axis UD in a clockwise direction around the center axis O, and the fourth turning axis IA2 is inclined by θ4 (=90°−θ1) with respect to the second turning axis UD in a counterclockwise direction around the center axis O.

Therefore, when an external force is applied to the coupled passive bending portion 15 which is in a straight state along the center axis O as shown in FIG. 6, the passive bending portion 15 is bendable as shown in FIG. 7. Accordingly, the passive bending portion 15 is bendable in the up, down, left, and right directions and in the direction in which the up, down, left, and right directions are combined, that is, bendable by 360° around the center axis O.

When the passive bending portion 15 bends in the up, down, left, and right directions, the respective bending pieces 41 turn around axes of all the rivets 47a and 47b. In addition, when the passive bending portion 15 bends in the inclined direction other than the up, down, left, and right directions, the respective bending pieces 41 turn around the axis of either of the rivet 47a or 47b.

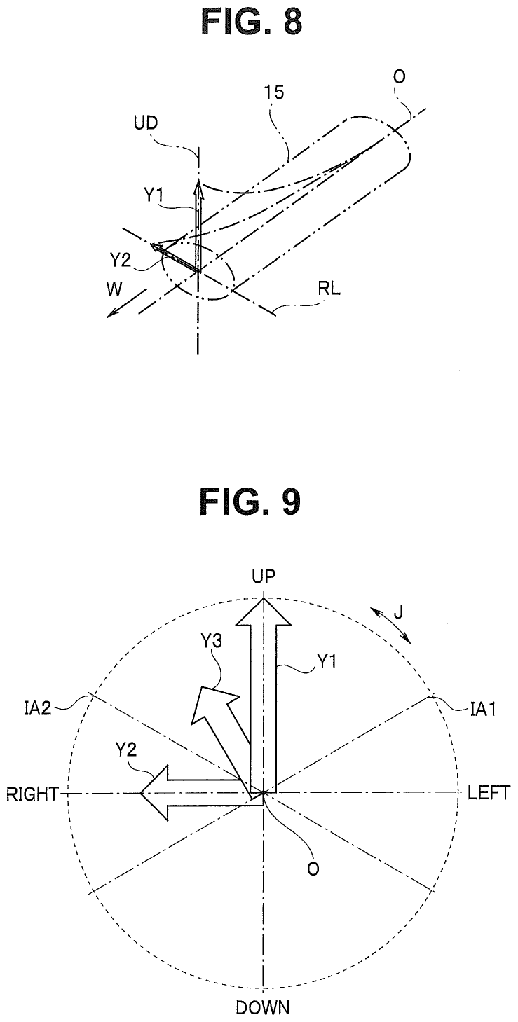

FIG. 8 is a view for describing a bendable range of the passive bending portion 15. FIG. 9 is a view schematically showing distribution of the maximum bending angle of the passive bending portion 15.

As shown in FIG. 8, the passive bending portion 15 is bendable by 360° around the center axis O in the insertion direction W. However, the passive bending portion 15 is configured in a manner that the plurality of bending pieces 41 are coupled as described above, and thus do not have the same maximum bending angle around the center axis O. As shown in FIG. 8, the passive bending portion 15 is bendable in the up-down direction other than in the left-right direction by the two turning axes IA1 and IA2 described above. In other words, bending rigidity in the left-right direction of the passive bending portion 15 is higher than the bending rigidity in the up-down direction, and the maximum bending angle in the left-right direction is smaller than the maximum bending angle in the up-down direction.

In other words, the passive bending portion 15 is a bending portion that is bent passively by the applied external force without being bent according to the bending operation by the operator, and has the higher bending rigidity in the second direction (left-right direction) orthogonal to the up-down direction rather than in the first direction (up-down direction). The second direction is parallel to the left-right direction orthogonal to the up-down direction of the image obtained by the image pickup unit 21 and displayed on the screen of the display apparatus.

In the present embodiment, as shown in FIG. 9, it is geometrically known that a maximum bending angle Y1 in the up-down direction of the passive bending portion 15 is 1.73 times a maximum bending angle Y2 in the left-right direction, that is, Y1=1.73 Y2.

Further, a maximum bending angle Y3 in an inclined direction is geometrically equal to the maximum bending angle Y2. In other words, the maximum bending angle Y1 in the up-down direction used most frequently for endoscope insertion into the large intestine is largest in the circumferential direction J, and the maximum bending angles in the other directions, i.e. the left-right direction and the inclined direction, are smaller than the maximum bending angle in the up-down direction.

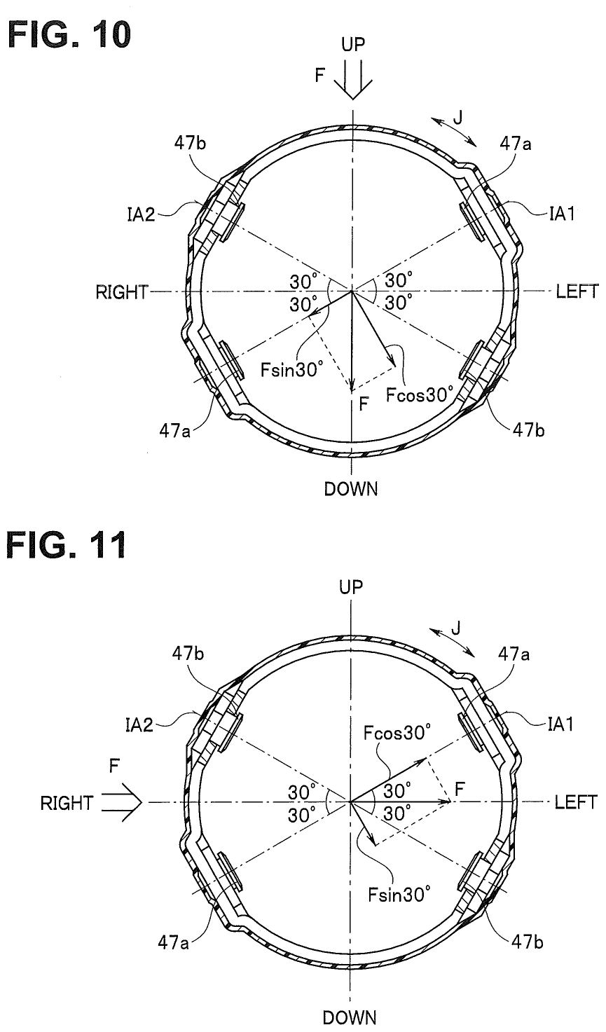

FIG. 10 is a view showing a component force applied to the turning axis when pressing of the large intestine is applied to the passive bending portion 15 of the present embodiment from the up-direction side. FIG. 11 is a view showing a component force applied to the turning axis when pressing of the large intestine is applied to the passive bending portion 15 of the present embodiment from the right-direction side.

As shown in FIG. 10, when an external force F is applied from the up-direction side of the passive bending portion 15 (including an external force from the down-direction side), a component force of F cos 30° acts on the bending of the passive bending portion 15 on the third turning axis IA1. The component force of F sin 30° is canceled because of acting on the same axis as the third turning axis IA1, and does not affect the bending of the passive bending portion 15. Although not being shown in FIG. 10, the component force described above also acts on the fourth turning axis IA2.

On the other hand, as shown in FIG. 11, when an external force F is applied from the right-direction side of the passive bending portion 15 (including an external force from the left-direction side), a component force of F sin 30° acts on the bending of the passive bending portion 15 on the third turning axis IA1. The component force of F cos 30° is canceled because of acting on the same axis as the third turning axis IA1, and does not affect the bending of the passive bending portion 15. Although not being shown in FIG. 11, the component force described above also acts on the fourth turning axis IA2.

In other words, the respective component forces applied to third turning axis IA1 and the fourth turning axis IA2 differ from each other when the external force is applied in the up-down direction and the external force is applied in the left-right direction, a relationship of F cos 30°>F sin 30° is satisfied, and thus the bending rigidity of the passive bending portion 15 is higher in the left-right direction rather than in the up-down direction. In other words, the passive bending portion is difficult to bend in the left-right direction.

As described above, the passive bending portion 15 does not bend at all in the left-right direction, and is configured to be bendable even in the inclined direction up to the maximum bending angle Y3 that is equal to or substantially equal to the maximum bending angle Y2 in the left-right direction.

(Operation)

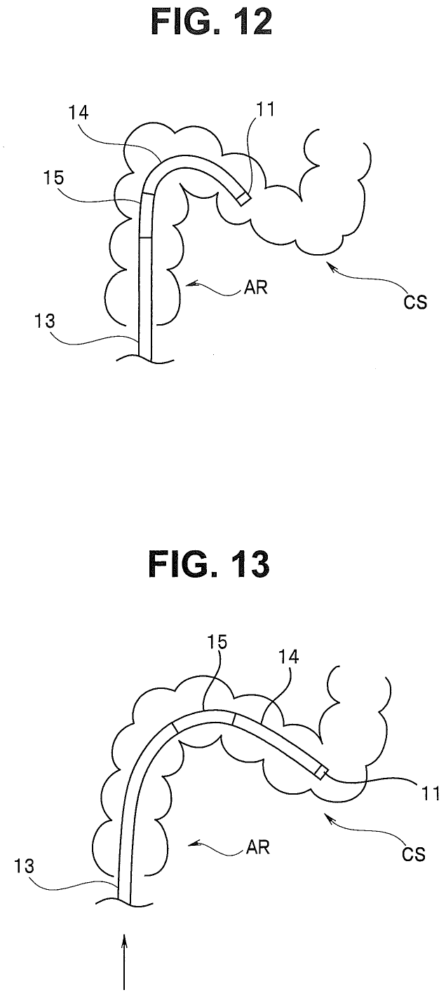

An operation of inserting the insertion portion 2 having the above-described configuration into the subject will be described. Here, an operation of inserting the insertion portion 2 into the large intestine will be described. FIGS. 12 and 13 are views for describing an operation of inserting the insertion portion 2 into the large intestine using the endoscope of the present embodiment.

When the operator inserts the distal end portion 11 of the insertion portion 2 from the rectum AR to the site of the sigmoid colon CS, the operators pushes the insertion portion 2 while bending the active bending portion 14 in any of the up-down directions used most frequently for insertion into the large intestine. When the active bending portion 14 of the insertion portion 2 enters the sigmoid colon CS from the rectum AR of the large intestine, the passive bending portion 15 is easily bent in the up-down direction similar to the active bending portion 14 by pressing from the intestinal wall.

As described above, since the passive bending portion 15 has the largest maximum bending angle in the circumferential direction J with respect to the up-down direction and further has low rigidity, the insertion portion 2 can enter the sigmoid colon CS, which is a flexure portion, without arising a known knocking-up phenomenon as shown in FIG. 13, and thus the burden and pain are reduced.

Further, even if the passive bending portion 15 is pressed from the intestinal wall in a direction slightly deviated from the up-down direction side when the insertion portion 2 is further pushed in and enters the sigmoid colon CS of the large intestine, since the bending rigidity is larger in the left-right direction, the passive bending portion 15 is bent in the up-down direction as shown in FIG. 13, and the insertion portion 2 can be smoothly advanced in the flexure portion without causing the operator to feel strange.

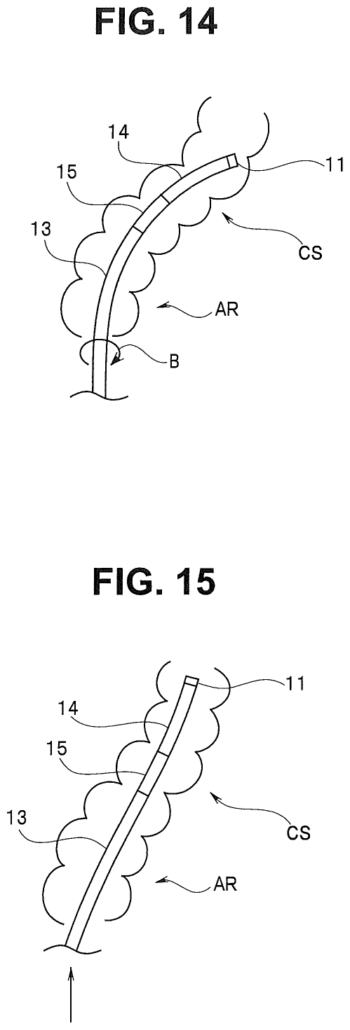

FIGS. 14 and 15 are views for describing an operation of straightening the large intestine using the endoscope of the present embodiment.

When operator straightens the large intestine, the operator swings the insertion portion 2 clockwise in the advancing direction as indicated by an arrow B in FIG. 14. Due to the swinging of the insertion portion 2, the passive bending portion 15 is pressed from the intestinal wall in the left-right direction. However, since the passive bending portion 15 has higher bending rigidity in the left-right direction, the passive bending portion 15 is difficult to bend in the left-right direction. Accordingly, the passive bending portion 15 does not easily bend in the left-right direction contrary to the operator's intention, and the large intestine is straightened by the insertion portion 2 as shown in FIG. 15.

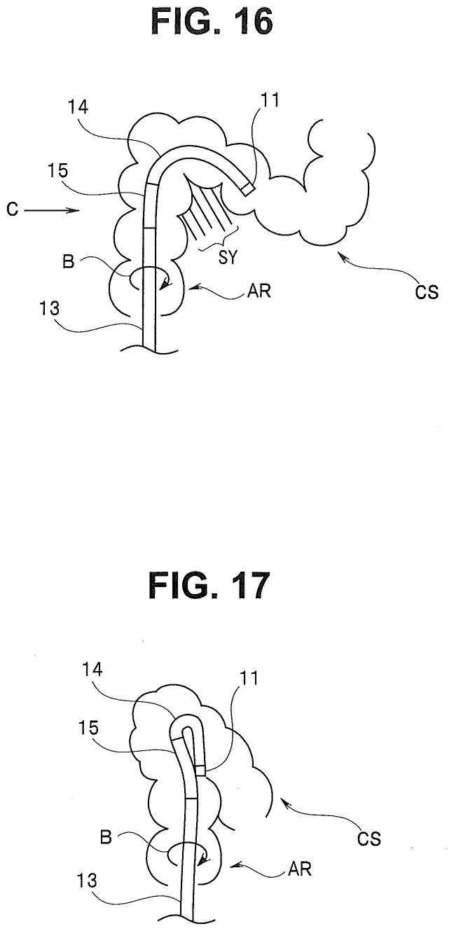

In addition, mobility of the large intestine may be lowered. For example, as shown in FIG. 16, when the mobility of the large intestine is lowered due to slight adhesion (SY) or the like, the pressing increases from the large intestine during the straightening of the large intestine. FIGS. 16 and 17 are views for describing a case where the mobility of the large intestine is lowered. FIG. 17 shows a state of the large intestine and the insertion portion as viewed from an arrow C in FIG. 16.

When the mobility of the large intestine is lowered, since the pressing increases from the large intestine as described above, the passive bending portion 15 is bent in the left-right direction. However, as shown in FIG. 17, since the maximum bending angle in the left-right direction of the passive bending portion 15 is small, the large intestine can be straightened without causing the operator to feel strange. Therefore, even when the mobility of the large intestine is lowered, the flexure portion can be easily straightened.

In the above-described embodiment, the third turning axis IA1 and the fourth turning axis IA2 of the passive bending portion 15 are respectively set on the positions deviated by +30° and −30° in the circumferential direction J from the first turning axis RL of the active bending portion 14, but the angles of the third turning axis IA1 and the fourth turning axis IA2 are not limited to the angles. As described above, similar operation and effect can be obtained as long as the angles of the third turning axis and the fourth turning axis are in the range of from an angle greater than 0° to an angle less than +45° and in the range of from an angle greater than −45° to an angle less than 0°, respectively.

Further, when the third turning axis IA1 and the fourth turning axis IA2 are respectively set in the range of from about +20° to +40° and in the range of from about −20° to −40° in the circumferential direction J from the first turning axis RL of the active bending portion 14, the maximum bending angle in the left-right direction and the maximum bending angle in the inclined direction of the passive bending portion 15 are substantially equal to each other (most preferably, +30° and)−30°, and the passive bending portion 15 is bent at a substantially constant maximum bending angle even when the insertion portion 2 is pushed while the active bending portion 14 is bent in any direction. Accordingly, the above-described effects can be exhibited without significantly reducing pass-ability of the insertion portion 2 in the flexure portion of the subject such as the sigmoid colon, that is, flexure pass-ability.

As described above, according to the present embodiment, it is possible to achieve the insertion device in which the passive bending portion is hardly bent in an unintended direction when the insertion portion is inserted into the subject and passes through the flexure portion.

### Second Embodiment

In the first embodiment, when the insertion portion 2 is viewed from the distal end, the passive bending portion 15 includes the third and fourth turning axes IA1 and IA2, the third turning axis IA1 being inclined by less than 45° with respect to the second turning axis RL in a counterclockwise direction around the center axis O, and the fourth turning axis IA2 being inclined by less than 45° with respect to the second turning axis RL in a clockwise direction around the center axis O. On the other hand, a passive bending portion 15 of the second embodiment further includes a fifth turning axis.

(Configuration)

Since a configuration of an endoscope according to the second embodiment is substantially similar to the configuration of the endoscope according to the first embodiment, components of the present embodiment similar to the components of the first embodiment are denoted by the same reference numerals and will not be described below, and only different configurations will be described.

The endoscope of the present embodiment has the configuration shown in FIGS. 1 and 2, and the active bending portion 14 has the configuration shown in FIG. 3.

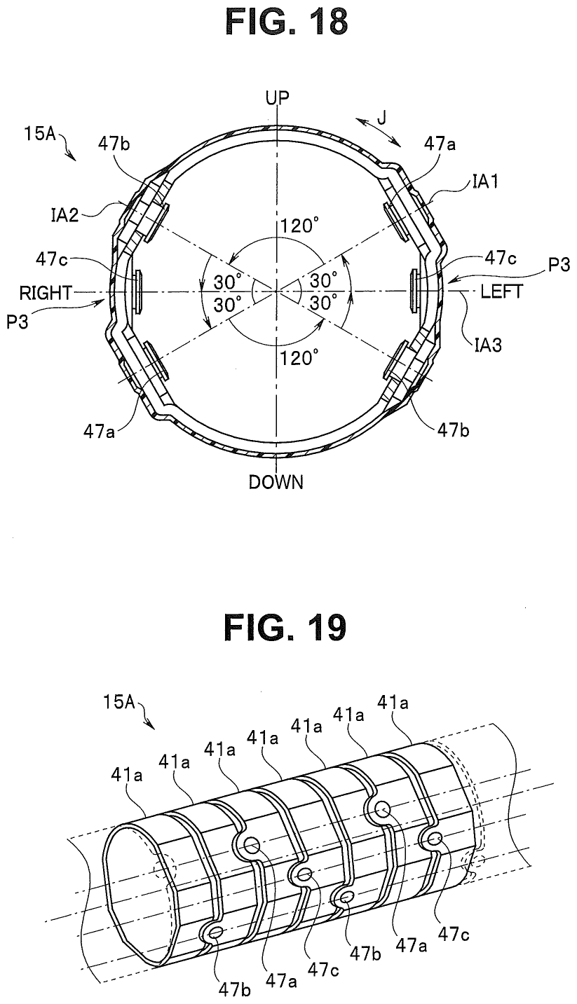

FIG. 18 is a cross-sectional view of a passive bending portion 15A as viewed from the distal end side of the insertion portion 2. FIG. 18 is a view as viewed in a direction of an arrow A in FIG. 4. FIG. 19 is a perspective view of a plurality of bending pieces of the passive bending portion 15A which is in a straight state.

The passive bending portion 15A of the present embodiment includes two fifth turning axes IA3 parallel to the first turning axis RL of the active bending portion 14 in addition to the third turning axis IA1 and the fourth turning axis IA2 of the first embodiment.

The passive bending portion 15A includes a plurality of bending pieces 41a. As shown in FIG. 19, the plurality of bending pieces 41a are coupled in the insertion direction W so that the passive bending portion 15A is bendable. Note that a length of each of the bending pieces 41a in the insertion direction W is shorter than the length of the bending piece 41 of the first embodiment. The reason is to prevent the length of the passive bending portion 15A from increasing because the number of the bending pieces 41a in the passive bending portion 15A is larger than the number of the bending pieces 41.

Specifically, in the passive bending portion 15A, the two adjacent bending pieces 41a in the insertion direction W are coupled to each other at two positions P1 on the third turning axis IA1, which is inclined by a predetermined first angle θ1 (here, 30°) with respect to the first turning axis RL around the center axis O of the insertion portion 2, by two rivets 47a, or at two positions P2 on the fourth turning axis IA2, which is inclined by a predetermined second angle θ2 (here, −30°) with respect to the first turning axis RL around the center axis O of the insertion portion 2, by two rivets 47b. In other words, the third turning axis IA1 and the fourth turning axis IA2 are similar to the third turning axis IA1 and the fourth turning axis IA2 of the first embodiment.

Further, the passive bending portion 15A includes two bending pieces 41 in which two adjacent bending pieces 41a in the insertion direction W are coupled at two positions on the fifth turning axis IA3 by two rivets 47c.

In other words, the passive bending portion 15A includes two adjacent bending pieces 41a which are coupled to each other on the third turning axis IA3 parallel to the first turning axis RL when the insertion portion 2 is viewed from the distal end side, among the plurality of bending pieces 41a.

The third turning axis IA1 is inclined by the predetermined first angle θ1 (here, 30°) with respect to the first turning axis RL when the passive bending portion 15A is viewed from the distal end side of the insertion portion 2. The fourth turning axis IA2 is inclined by the predetermined second angle θ2 (here, −30°) with respect to the first turning axis RL. The fifth turning axis IA3 is parallel to the first turning axis RL. As shown in FIG. 19, the passive bending portion 15A includes two third turning axes IA1, two fourth turning axes IA2, and two fifth turning axes.

Then, the bending piece 41a on the proximal end side, which is one of the bending pieces 41a coupled by the two rivets 47a, is coupled to the adjacent bending piece 41a on the proximal end side by two rivets 47b at a position on the fourth turning axis IA2 turned by 120° counterclockwise around the center axis O from the third turning axis IA1 when the passive bending portion 15A is viewed from the distal end side of the insertion portion 2.

The bending piece 41 on the proximal end side, which is one of the bending pieces 41a coupled by the two rivets 47b, is coupled to the adjacent bending piece 41a on the proximal end side by two rivets 47c at a position on the fifth turning axis IA3 turned by 30° counterclockwise around the center axis O from the fourth turning axis IA2 when the passive bending portion 15A is viewed from the distal end side of the insertion portion 2.

The bending piece 41 on the proximal end side, which is one of the bending pieces 41a coupled by the two rivets 47c, is coupled to the adjacent bending piece 41a on the proximal end side by two rivets 47a at a position on the third turning axis IA1 turned by 30° counterclockwise around the center axis O from the fifth turning axis IA3 when the passive bending portion 15A is viewed from the distal end side of the insertion portion 2.

In other words, the coupling of the two adjacent bending pieces 41a on the fifth turning axis IA3 is located between the coupling of the two adjacent bending pieces 41a on the third turning axis IA1 and the coupling of the two adjacent bending pieces 41a on the fourth turning axis IA2. The plurality of bending pieces 41a of the passive bending portion 15A are coupled so as to have such a coupling relationship.

(Operation)

When the insertion portion 2 including the passive bending portion 15A of the present embodiment is inserted into the subject, the operator pushes the insertion portion 2 while bending the active bending portion 14 in any of the up-down direction to straighten the flexure portion of the sigmoid colon or the like as described in the first embodiment, thereby the insertion portion 2 passes through the flexure portion of the sigmoid colon or the like.

At that time, the maximum bending angle Y1 is preferably large in the up-down direction, but, as described above in the first embodiment, the maximum bending angle Y1 in the up-down direction of the passive bending portion 15 and the maximum bending angle Y2 in the left-right direction have a geometrical relationship of Y1=1.73 Y2. In other words, when the passive bending portion 15 is configured by only two axes of the third turning axis IA1 and the fourth turning axis IA2, if the maximum bending angle Y1 in the up-down direction is set large, Y2 necessarily also increases.

The maximum bending angle Y2 in the left-right direction is preferably small at the time of straightening the flexure portion. However, the maximum bending angle Y2 in the left-right direction necessarily also increases for improvement in flexure pass-ability in the up-down direction, and the passive bending portion 15 may be bent carelessly.

On the other hand, the passive bending portion 15A of the present embodiment includes the fifth turning axis IA3, so that the maximum bending angle Y1 in the up-down direction and the maximum bending angle Y2 in the left-right direction can be independent and arbitrarily set. Therefore, even when the maximum bending angle Y1 in the up-down direction is set large, the maximum bending angle Y2 in the left-right direction does not necessarily increase.

As described above, according to the present embodiment, it is possible to achieve the insertion device in which the passive bending portion is hardly bent in an unintended direction when the insertion portion is inserted into the subject and passes through the flexure portion.

### Third Embodiment

The passive bending portion includes the plurality of bending pieces in the first and second embodiments, but a passive bending portion of a third embodiment does not include a plurality of bending pieces.

(Configuration)

Since a configuration of an endoscope according to the third embodiment is substantially similar to the configuration of the endoscope according to the first embodiment, components of the present embodiment similar to the components of the first embodiment are denoted by the same reference numerals and will not be described below, and only different configurations will be described.

The passive bending portion, which is the first flexible tube portion, is configured by the plurality of bending pieces in the two embodiments described above, but may have a configuration similar to a corrugated tube 13 including an outer cover and a helical tube as will be described below in the present embodiment.

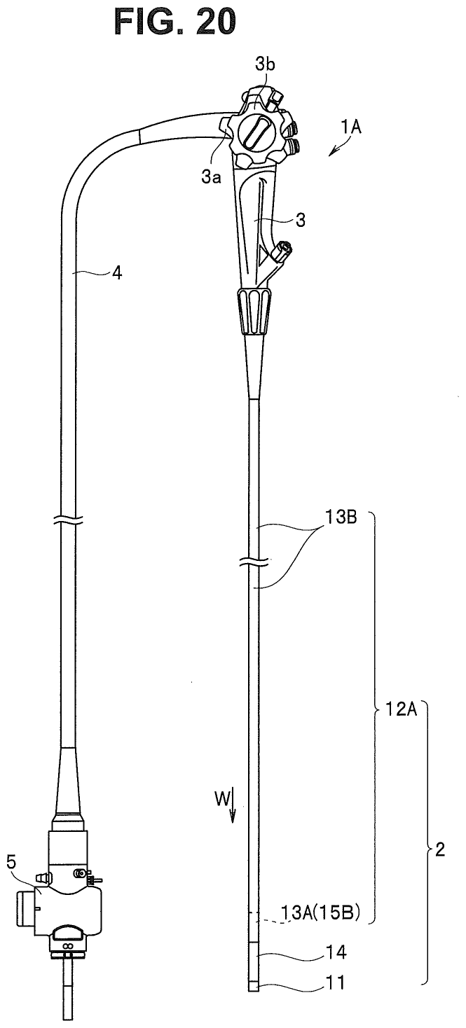

FIG. 20 is a schematic diagram of an endoscope 1A of the present embodiment. An insertion portion 2 of the endoscope 1A includes a distal end portion 11, an active bending portion 14, and a flexible tube portion 12A in order from a distal end, and is formed in an elongated shape in an insertion direction W.

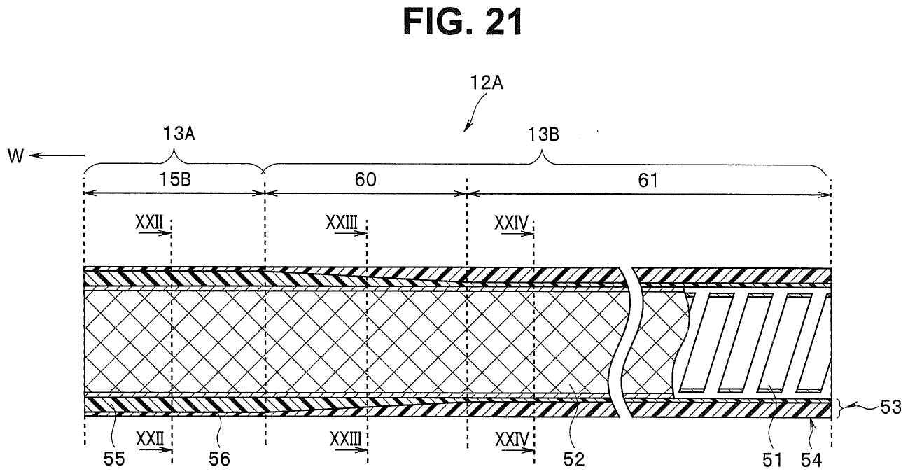

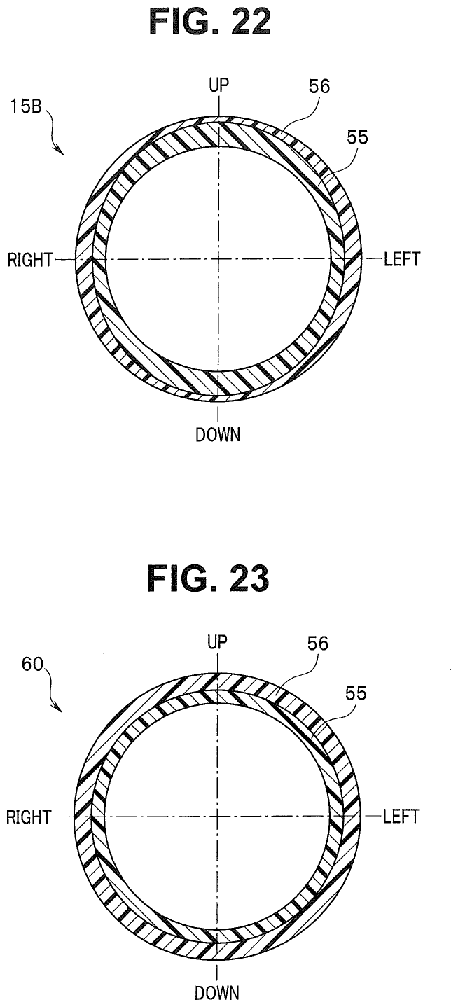

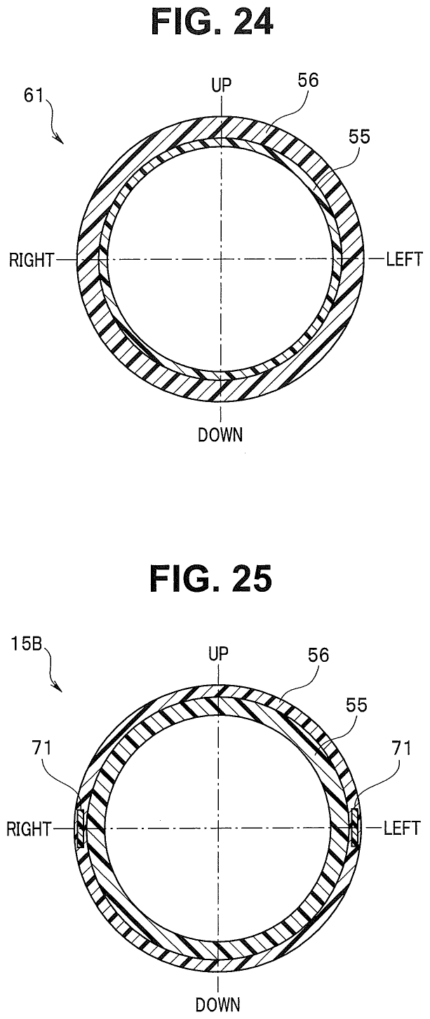

FIG. 21 is a cross-sectional view of the flexible tube portion 12A taken along a center axis O in an up-down direction. FIG. 22 is a cross-sectional view of the flexible tube portion 12A taken along line XXII-XXII in FIG. 21. FIG. 23 is a cross-sectional view of the flexible tube portion 12A taken along line XXIII-XXIII in FIG. 21. FIG. 24 is a cross-sectional view of the flexible tube portion 12A taken along line XXIV-XXIV in FIG. 21. Note that a helical tube 51, a mesh tube 52, and various internal components are not shown in FIGS. 22 to 24.

The active bending portion 14 is provided on a distal end side relative to the flexible tube portion 12A. The flexible tube portion 12A has a hollow shape, and a plurality of signal lines and a plurality of bending wires 35a to 35d are inserted into the flexible tube portion 12A. As shown in FIG. 21, the flexible tube portion 12A includes a helical tube 51 formed by helically winding a strip-shaped thin plate member, a mesh-like mesh tube 52 that is provided on an outer peripheral surface of the helical tube 51, and an outer cover 53 that is provided on an outer peripheral surface of the mesh tube 52.

On the outer peripheral surface of the outer cover 53, a coating layer 54 is provided in which a fluorine-containing coating agent having chemical resistance are laminated.

The outer cover 53 is a tubular member having, for example, a double-layered configuration in which a flexible resin layer 55 configured to cover the outer peripheral surface of the mesh tube 52 and a rigid resin layer 56 configured to cover the outer peripheral surface of the flexible resin layer 55 are laminated.

The flexible resin layer 55 is made of a flexible resin, and the rigid resin layer 56 is made of a rigid resin and is harder than the flexible resin layer 55. Examples of the resin used for the flexible resin layer 55 and the rigid resin layer 56 include two kinds of thermoplastic urethane elastomers having different types of hardness.

The flexible tube portion 12A includes a first corrugated tube 13A, which is a first flexible tube portion (15B), and a second corrugated tube 13B, which is a second flexible tube portion in order from the distal end. The second corrugated tube 13B includes a distal end-side portion 60 and a proximal end-side portion 61.

The first corrugated tube 13A configures as a whole a thick flexible portion in which a thickness of the flexible resin layer 55 is larger than a thickness of the rigid resin layer 56. In the first corrugated tube 13A, as shown in FIG. 22, the flexible resin layer 55 has the thickness smaller in the left-right direction than in the up-down direction.

In the second corrugated tube 13B, the distal end-side portion 60 configures a flexibility changing portion in which a thickness ratio of the flexible resin layer 55 to the rigid resin layer 56 changes. In the distal end-side portion 60 of the second corrugated tube 13B, the flexible resin layer 55 and the rigid resin layer 56 are formed such that the thickness of the flexible resin layer 55 is gradually smaller than the thickness of the rigid resin layer 56 from the distal end toward proximal end and the thickness of the rigid resin layer 56 is gradually larger than the thickness of the flexible resin layer 55 from the distal end toward the proximal end.

In the second corrugated tube 13B, the proximal end-side portion 61 configures a flexible portion in which the thickness of the rigid resin layer 56 is larger than the thickness of the flexible resin layer 55.

A thickness of the outer cover 53 including the flexible resin layer 55 and the rigid resin layer 56 is the same in the first corrugated tube 13A and the distal end-side portion 60 and the proximal end-side portion 61 in the second corrugated tube 13B.

In particular, as shown in FIG. 22, the flexible resin layer 55 and the rigid resin layer 56 in the first corrugated tube 13A are different in thickness ratio from each other in the up-down direction and the left-right direction. The rigid resin layer 56 is thicker in the left-right direction than the flexible resin layer 55 so that the bending rigidity is higher in the left-right direction rather than in the up-down direction. In other words, the first corrugated tube 13A includes a tubular member having higher bending rigidity in the left-right direction than in the up-down direction. Here, the thickness of the flexible resin layer 55 serving as a thin portion of the tubular member differs from the thickness of the rigid resin layer 56 in the up-down direction and the left-right direction when the insertion portion 2 is viewed from the distal end side, so that the flexible resin layer 55 and the rigid resin layer 56, which are tubular members, have higher bending rigidity in the left-right direction than in the up-down direction.

In the distal end-side portion 60 and the proximal end-side portion 61 of the second corrugated tube 13B, the thickness ratio of the flexible resin layer 55 is equal to the thickness ratio of the rigid resin layer 56 in the up-down direction and the left-right direction.

As described above, the first corrugated tube 13A configures the passive bending portion provided on the proximal end side of the active bending portion 14, as the first flexible tube portion 15B.

(Operation)

As described above, since the rigidity of the first corrugated tube 13A is higher in the left-right direction than in the up-down direction, the first corrugated tube 13A is hardly bent in the left-right direction rather than the up-down direction. Accordingly, the first corrugated tube 13A (passive bending portion 15B) functions in the same manner as the passive bending portions 15 and 15A of the first and second embodiments, and the operator can smoothly pass the insertion portion 2 through the flexure portion of the subject.

(Modification 1)

As Modification 1 of the third embodiment, instead of making the thickness of the thin portion in the up-down direction of the flexible resin layer 55 and the rigid resin layer 56 of the first corrugated tube 13A different from the thickness of the thin portion in the left-right direction, a member having a hardness higher than the hardness of the rigid resin layer 56, for example, an elongated piece 71 made of resin, may be embedded in the rigid resin layer 56 in parallel to the center axis O so as to increase the bending rigidity in the left-right direction.

FIG. 25 is a cross-sectional view of a first corrugated tube 13A according to Modification 1, taken along line XXII-XXII in FIG. 21. As shown in FIG. 25, the strip-shaped elongated piece 71 having a higher hardness than the rigid resin layer 56 is disposed in the rigid resin layer 56 so that a longitudinal axis of the elongated piece 71 is parallel to the center axis O. Two elongated pieces 71 are disposed symmetrically with respect to the center axis O.

In order to increase the bending rigidity in the left-right direction, the elongated piece 71 may be embedded in the flexible resin layer 55. In other words, the flexible resin layer 55 or the rigid resin layer 56 serving as a tubular member includes two members having higher bending rigidity in the left-right direction than in the up-down direction on an axis in the left-right direction when the insertion portion 2 is viewed from the distal end.

Furthermore, the elongated piece 71 may be a fiber member that is difficult to extend and contract.

Accordingly, even when the thickness of the flexible resin layer 55 and the thickness of the rigid resin layer 56 in the first corrugated tube 13A are equal to each other, the first corrugated tube 13A is difficult to bend in the direction of the first turning axis RL that is the left-right direction due to the two strip-shaped elongated pieces 71 having higher hardness.

As described above, according to the embodiments and Modification, it is possible to achieve the insertion device in which the passive bending portion is difficult to bend in an unintended direction when the insertion portion is inserted into the subject and passes through the flexure portion.

The first corrugated tube 13A and the second corrugated tube 13B are integrally formed in the embodiments, but two separate corrugated tubes each having a structure including the helical tube 51, the mesh tube 52, and the outer cover 53 may be integrally formed to be consecutively connected.

(Procedure)

A procedure of inserting the insertion portion into the sigmoid colon of the large intestine using the endoscope according to the above-described three embodiments will be described below.

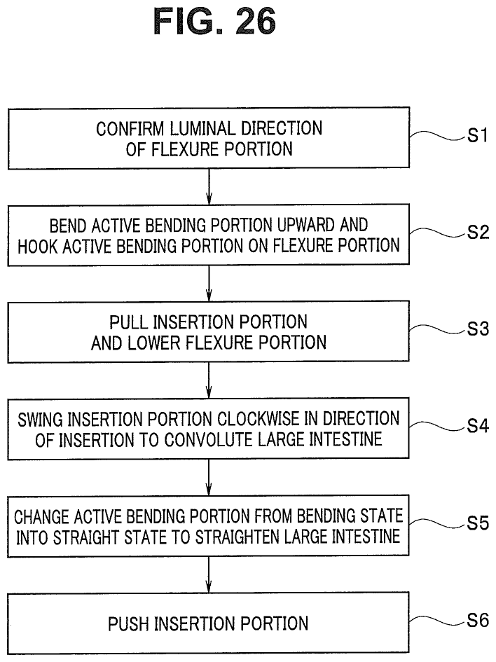

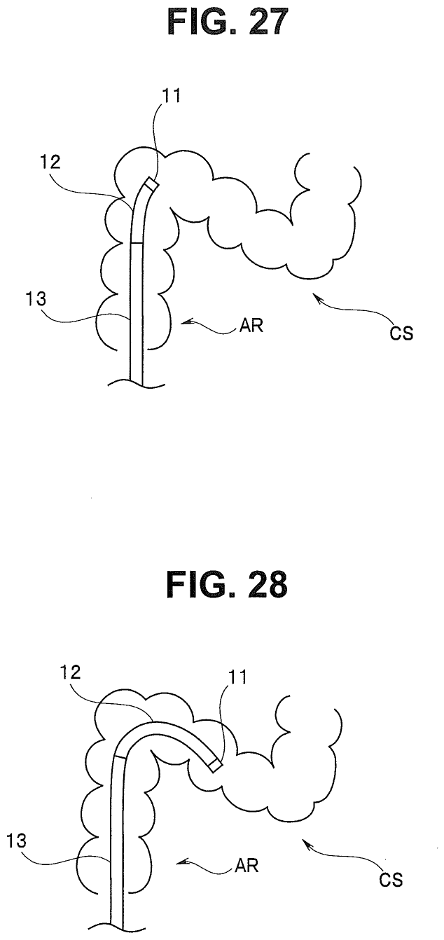

FIG. 26 is a flowchart showing an example of a procedure in which straightening of the flexure portion of the subject is performed. FIGS. 27 to 32 are views showing examples of a state of the insertion portion inserted into the large intestine.

The examiner inserts the distal end portion 11 of the insertion portion 2 from the anus, and confirms a luminal direction of the sigmoid colon CS which is a flexure portion (step (hereinafter abbreviated as S) 1). As shown in FIG. 27, the examiner can confirm the luminal direction of the sigmoid colon CS by directing the distal end portion 11 toward the entrance of the sigmoid colon CS.

Subsequently, the examiner bends the active bending portion 14 upward and hooks the active bending portion 14 on the flexure portion (S2). As shown in FIG. 28, the active bending portion 14 is hooked on the sigmoid colon CS.

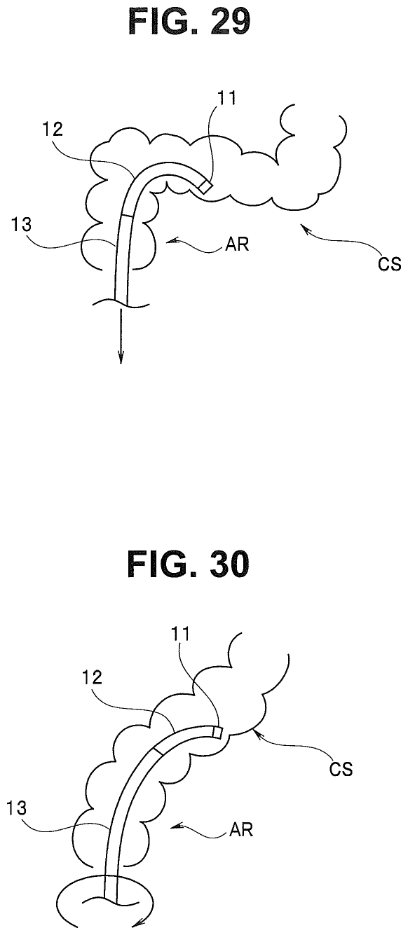

Then, the examiner pulls the insertion portion 2 and lowers the flexure portion (S3). As shown in FIG. 29, as the sigmoid colon CS approaches the examiner, the entrance of the sigmoid colon CS is lowered.

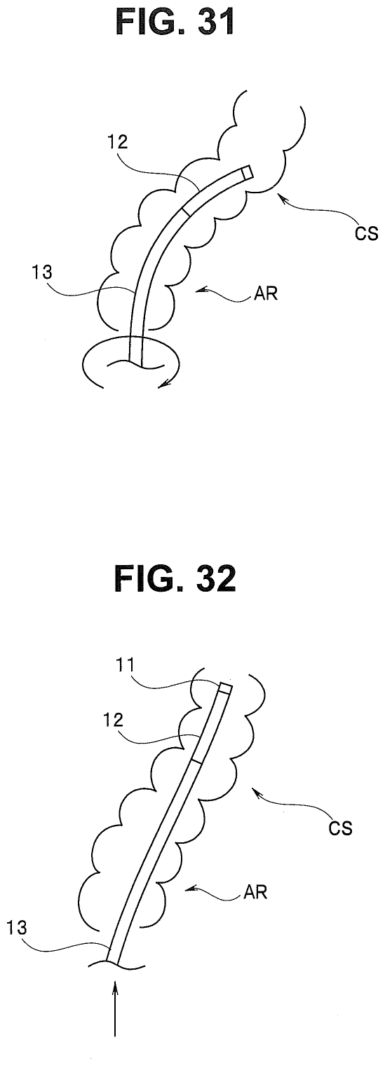

When the examiner swings the insertion portion 2 clockwise in the insertion direction, the bending portion 12 rises up and the large intestine is convoluted as shown in FIG. 30 (S4). FIG. 31 shows a state where the large intestine is convoluted.

Then, the examiner changes the active bending portion 14 from the bending state into a straight state to straighten the large intestine (S5). FIG. 32 shows a state where the large intestine is straightened.

When the large intestine is straightened, the examiner can push the insertion portion deeply (S6).

As described above, according to the respective embodiments described above, it is possible to achieve the insertion device in which the passive bending portion is hardly bent in an unintended direction when the insertion portion is inserted into the subject and passes through the flexure portion.

As a result, the operator can smoothly pass the insertion portion 2 through the flexure portion of the subject.

The present invention is not limited to the above-described embodiments, and various changes and modifications can be made without departing from the scope of the present invention.

What is claimed is:

1. An insertion device comprising an insertion portion configured to be inserted into a subject from a distal end side in a longitudinal axis direction,
the insertion portion including:
a distal end portion provided at a distal end of the insertion portion;
a bending portion provided on a proximal end side of the distal end portion and configured to bend in a first direction according to a bending operation by an operator;
a passive bending portion provided on a proximal end side of the bending portion and configured to passively bend by receiving an external force without being bent by the bending operation by the operator; and
a flexible tube portion provided on a proximal end side of the passive bending portion and having flexibility, wherein
the passive bending portion includes a plurality of annular bending pieces connected in series, and two adjacent bending pieces in the plurality of bending pieces are coupled to each other on either of a first turning axis inclined by a first angle being in a range of from an angle greater than 0° to an angle less than +45° in a second direction orthogonal to the first direction around a center axis of the insertion portion or a second turning axis inclined by a second angle being in a range of from an angle greater than −45° to an angle less than 0° when the insertion portion is viewed from the distal end side, so that the passive bending portion has higher bending rigidity in the second direction than in the first direction.

2. The insertion device according to claim 1, wherein
the first angle is in a range of from an angle greater than 0° to an angle +30°, and
the second angle is in a range of from −30° to an angle less than 0°.

3. The insertion device according to claim 1, wherein
first coupling of the two adjacent bending pieces on the first turning axis and second coupling of the two adjacent bending pieces on the second turning axis are alternately performed in the longitudinal axis direction.

4. The insertion device according to claim 3, wherein
the passive bending portion includes two adjacent bending pieces, which are coupled to each other on a third turning axis parallel to the second direction when the insertion portion is viewed from the distal end side, among the plurality of bending pieces.

5. The insertion device according to claim 4, wherein
third coupling of the two adjacent bending pieces on the third turning axis is located between the first coupling and the second coupling.

6. The insertion device according to claim 1, wherein
the distal end portion is provided with an image acquisition apparatus configured to pick up an image of the subject, and
the second direction is parallel to a left-right direction orthogonal to an up-down direction of an image obtained by the image acquisition apparatus and displayed on a screen of a display apparatus.

7. The insertion device according to claim 1, wherein
the bending portion is bendable also in the second direction when the insertion portion is viewed form the distal end side, according to the bending operation by the operator.

8. The insertion device according to claim 1, wherein
an inclined angle of the first turning axis and an inclined angle of the second turning axis are set to be equal to each other in the second direction.

9. An insertion device comprising an insertion portion configured to be inserted into a subject from a distal end side in a longitudinal axis direction,
the insertion portion including:
a distal end portion provided at a distal end of the insertion portion;
a bending portion provided on a proximal end side of the distal end portion and configured to bend in a first direction and a second direction orthogonal to the first direction when the insertion portion is viewed from the distal end side, according to a bending operation by an operator;
a passive bending portion provided on a proximal end side of the bending portion and configured to passively bend by receiving an external force without being bent by the bending operation by the operator;
a flexible tube portion provided on a proximal end side of the passive bending portion and having flexibility; and
an image acquisition apparatus provided at the distal end portion and configured to pick up an image of the subject, wherein
the passive bending portion includes a plurality of annular bending pieces connected in series, two adjacent bending pieces in the plurality of bending pieces are coupled to each other on either of a first turning axis inclined by a first angle being in a range of from an angle greater than 0° to an angle +30° in the second direction around a center axis of the insertion portion or a second turning axis inclined by a second angle being in a range of from −30° to an angle less than 0° when the insertion portion is viewed from the distal end side, so that the passive bending portion has higher bending rigidity in the second direction than in the first direction, an inclined angle of the first turning axis and an inclined angle of the second turning axis are set to be equal to each other in the second direction, and the second direction is parallel to a left-right direction orthogonal to an up-down direction of an image obtained by the image acquisition apparatus and displayed on a screen of a display apparatus.
